(19) United States
(12) Patent Application Publication
(10) Pub. No.: US 2006/0040871 A1
(43) Pub. Date: Feb. 23, 2006

(54) Bacterial transforming agent

(76) Inventors: Michael Ernest Levey, West Sussex (GB); Robert Leslie Rowland Hill, Surrey (GB)

Correspondence Address: YOUNG LAW FIRM;A PROFESSIONAL CORPORATION, 4370 ALPINE ROAD SUITE 106, PORTOLA VALLEY, CA, 94028, US

(21) Appl. No.: 10/517,359

(22) PCT Filed: Jun. 2, 2003
(86) PCT No.: PCT/GB03/02402

§ 371 Date: Jul. 27, 2005

(51) Int. Cl. A61K 31/198; A61K 38/04; A61K 31/16

(52) U.S. Cl. 514/19; 514/563; 514/616

(57) ABSTRACT

A method of increasing the sensitivity of a bacterial strain to a cell-wall active antimicrobial agent, to which the bacterial strain or a progenitor strain from which the bacterial strain has evolved is sensitive is described. The method comprises the step of exposing said bacterial strain to a transforming agent having the following formula (1): where moieties R₁, and R₂ are each independently selected from alkyl, alkyloxy, alkyloxycarbonyl, alkylcarbonyloxy, alkenyl, alkenyloxy, alkenyloxycarbonyl, alkenylcarbonyloxy, alkynyl, alkynyloxy: alkynyloxycarbonyl, alkynylcarbonyloxy, each of which may be substituted or unsubstituted, straight chain or branched or cyclic, aryl, aryloxy, aryloxycarbonyl, arylcarbonyloxy, each of which may be substituted or unsubstituted and cabamoyl, moiety R₃ is selected from alkyl, alkyloxy, alkylcarbonyloxy, alkenyl, alkenyloxy, alkenylcarbonyloxy, alkynyl, alkynyloxy, alkynylcarbonyloxy, each of which may be substituted o unsubstituted, straight chain or branched or cyclic, aryl, aryloxy, arylcarbonyloxy, each of which may be substituted or unsubstituted, and carboxyl other than R₁, R₂, and R₃ are not all H, and Y is selected from a natural amino acid side chain. The use of an agent having the above formula in the manufacture of a medicament for increasing the sensitivity of a bacterial strain infecting, colonising or being carried by a patient, to an cell-wall active antimicrobial agent is also disclosed.

The present invention relates to agents for increasing the sensitivity of bacteria to anti-microbial agents and particularly, but not exclusively, to agents for transforming bacteria resistant to an antimicrobial agent into bacteria having increased sensitivity to that antimicrobial agent.

The global rise of bacteria and other microorganisms resistant to antibiotics and antimicrobials in general, poses a major threat to mankind. Deployment of massive quantities of antimicrobial agents into the human ecosphere during the past 60 years has introduced a powerful selective pressure for the emergence and spread of antimicrobial-resistant bacterial pathogens. Resistant organisms of special epidemiological importance, due to the preponderance of these pathogens to cause cross-infection in hospitals and other health care settings, include methicillin-resistant Staphylococcus aureus (MRSA) and other Gram-positive bacteria such as vancomycin-resistant enterococci (VRE) and Clostridium difficile, and Streptococcus pneumoniae which is becoming increasingly resistant to β-lactams and other antimicrobials, plus Gram-negative rods that produce extended spectrum β-lactamases. As there is resistance to every clinically available antibiotic, particularly amongst recent strains of epidemic MRSA (EMRSA), there is the prospect of a post-antibiotic era where current antimicrobial agents are ineffective.

Staphylococcus aureus

S. aureus is an important cause of community- and hospital-acquired infection and is the second most important cause of septicaemia after Escherichia coli and the second commonest cause of line-associated infection and continuous ambulatory peritoneal dialysis peritonitis. S. aureus is also a major cause of bone, joint and skin infection. Overall, S. aureus is the commonest bacterial pathogen in modern hospitals and communities. It is also one of the most antimicrobial resistant and readily transmissible pathogens which, on average, may be carried by about a third of the normal human population, thus facilitating world-wide spread of epidemic strains.

Colonisation is a prerequisite for carriage and infection and staphylococci are well known colonisers of skin, wounds and implantable devices. Carriage usually occurs on specific skin sites histologically associated with apocrine glands, mainly the anterior nares (picking area of the nose) and secondarily the axillae and perineum. It has been postulated that S. aureus is disseminated from the nose to the hands and thence to other body sites where infection can occur when breaks in the dermal surfaces, by vascular catheterisation or surgical incision, have occurred. Intranasal mupirocin is the mainstay for the eradication of nasal carriage of Methicillin-resistant S. aureus (MRSA), which are by nature multiply antibiotic resistant, during hospital outbreaks. In view of the increasing concern about S. aureus infection it is imperative that new and reliable treatments for the elimination of carriage of S. aureus, are sought.

By the early 1950s, resistance to penicillin, conferred by a penicillinase (=β-lactamase) born on transmissible plasmids, was common in strains of S. aureus acquired in hospitals. Alternative antimicrobial agents, namely tetracycline, streptomycin and the macrolides, were introduced, but resistance developed rapidly. The understanding of the chemistry of the β-lactam ring enabled the development of methicillin, a semisynthetic penicillinase-stable isoxazolyl penicillin. Methicillin and the subsequent development of other isoxazolyl semisynthetic agents such as flucloxacillin, cloxacillin and oxacillin, revolutionised the treatment of S. aureus infections.

MRSA were first detected in England in 1960 and have since become a well recognised cause of hospital-acquired infection world-wide. MRSA are resistant to all clinically available -lactams and cephalosporins and readily acquire resistant determinants to other antimicrobial agents used in hospital medicine. Selective pressure has ensured the rise and world-wide spread of MRSA. Outbreaks caused by ‘modern’ epidemic MRSA (EMRSA) in the UK began during the early 1980s with a strain subsequently characterised as EMRSA-1. There are now 17 epidemic types recognised in the UK and these have steadily risen in prevalence in England and Wales from 1-2% of reported blood and CSF isolates in 1989-92 to 31.7% in 1997. This rise reflects the increasing domination by epidemic strain types 15 and 16. EMRSA are very transmissible and variably acquire resistance to all antimicrobials in addition to those related to methicillin and the β-lactam ring. In addition to EMRSA, is that of serious skin infection associated with community-acquired MRSA (C-MRSA). This is a rapidly rising phenomenon, recently reported in the USA, UK and continental Europe. Lower respiratory tract infection has also been reported. Many of these C-MRSA produce a toxin referred to as PVL, which is a leukocydin associated with high mortality. Serious infection derived from the skin and from nasal carriage (such as community-acquired pneumonia) of MRSA can be prevented by the use of appropriate anti-staphylococcal topical antimicrobials.

Vancomycin-Resistance

S. aureus/MRSA

A further sinister development is the ability of some strains to acquire reduced or intermediate resistance to glycopeptides. Glycopeptide antibiotics, vancomycin in particular, have been the drugs of choice, and in many cases the only active agents, for treating infection with MRSA and other resistant Gram-positive bacteria such as enterococci. If MRSA are not controlled, then the clinical use of vancomycin or teicoplanin rises because of the increased number of wound and blood stream infections in hospitalised patients. Soon after Hiramatsu reported vancomycin-intermediate-resistant MRSA in Japan (Lancet 1997,350, pp 1670-3), than EMRSA-16 began to reduce its sensitivity to vancomycin in some clinical isolates from diabetic foot ulcers. A new epidemic strain, EMRSA-17, evolved on the south coast of England and has a prepoderancy for reduced susceptibility to vancomycin. It is now thought that this strain developed from EMRSA-5 and demonstrates that epidemic strains are continually evolving with even greater resistance and propensity to cause serious disease. The most serious development is that of MRSA with high-level resistance to vancomycin (VRSA). These have been reported from the USA and the strains carry genes identical to the vancomycin-resistance genes in VRE. The spread of VRSA seems inevitable and, if there are no suitable antimicrobial agents to control carriage and wound infection, then the continuation of routine surgery in affected institutions is likely to be unsustainable.

Enterococci

Enterococci, particularly Enterococcus faecium and E. faecalis, are primarily gut commensals but which can become opportunistic pathogens that colonise and infect immunocompromised hosts, such as liver transplant patients. Vancomycin-resistant E. faecium (VREF) emerged and have since become important nosocomial pathogens. Since vancomycin-resistant enterococci first emerged in South London and Paris in 1987, multiply antimicrobial resistant enterococci have been reported with increasing frequency in many countries. Indeed, E. faecium resistant to gentamicin, vancomycin and other agents, have caused infections for which no therapeutic agents had been available in the UK, although quinupristin/dalfopristin, which is active (MIC≦2 mg/L) against 86% of E. faeciuin isolates, has now been licensed. In the USA, the proportion of VREF among enterococci isolated from blood cultures increased from 0% in 1989 to 25.9% in 1999. Raw poultry meat appears to be a major source of VREF.

Whilst antimicrobial resistance is of global concern, the only method proposed to control and reduce resistance is by encouraging appropriate use of antimicrobial agents. However, expectations that prudent antibiotic use will deliver reversals in resistance trends should only be accepted with caution. The concept of transforming resistant strains into sensitive ones, with the object of restoring the use of previously established antimicrobial agents rather than develop new agents to which resistance will subsequently develop, has not been explored.

An object of the present invention is to provide a Bacterial Transforming Agent (BTA) for reversing (partially or wholly) the resistance of a bacterial cell to an antimicrobial agent.

Bacterial Transforming Agents are known and have the Following Characteristics

Where microorganisms have cell walls resistant to cell-wall-active antimicrobials and this resistance is reliant upon inter-cell-wall cross-links, BTAs transform the resistant microorganism from its resistant state to that of a sensitive one to the cell-wall-active agent.

The presence of a BTA is essential for transformation to occur.

BTAs are not therapeutic agents on their own, at the concentrations at which they are used as BTA's.

The effect of the BTA on the target microorganism is reversed when the BTA is removed.

BTAs are not inhibitors of a specific resistance mechanism, such as a β-lactamase, efflux pump or antibiotic-destroying enzyme.

The present invention resides in a method of increasing the sensitivity of a bacterial strain to an antimicrobial cell-wall active agent, to which the bacterial strain or a progenitor strain from which the bacterial strain has evolved is sensitive, said method comprising the step of exposing said bacterial strain to a transforming agent having the following formula (I):- 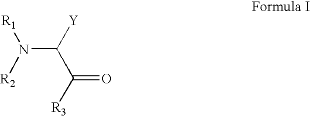
where

moieties R₁ and R₂ are each independently selected from, alkyl, alkyloxy, alkyloxycarbonyl, alkylcarbonyloxy, alkenyl, alkenyloxy, alkenyloxycarbonyl, alkenylcarbonyloxy, alkynyl, alkynyloxy, alkynyloxycarbonyl, alkynylcarbonyloxy, each of which may be substituted or unsubstituted, straight chain or branched or cyclic,

aryl, aryloxy, aryloxycarbonyl, arylcarbonyloxy, each of which may be substituted or unsubstituted, and

cabamoyl,

moiety R₃ is selected from alkyl, alkyloxy, alkylcarbonyloxy, alkenyl, alkenyloxy, alkenylcarbonyloxy, alkynyl, alkynyloxy, alkynylcarbonyloxy, each of which maybe substituted or unsubstituted, straight chain or branched or cyclic,

aryl, aryloxy, arylcarbonyloxy, each of which may be substituted or unsubstituted, and carboxyl.

other than R₁, R₂, and R₃ are not all H,

and Y is selected from a natural amino acid side chain.

Sulphur analogues of said oxygen containing substituents are also within the scope of the invention. Reference to cyclic compounds is intended to include heterocyclic compounds having one or more N, S or O atoms in their ring system.

Suitable substituents on any of said R₁, R₂ and R₃moieties include halogen (eg. F and Cl), hydroxyl (—OH), carboxyl (—CO₂H), amine and amide.

Preferably Y is —H₂ (i.e. glycine “side chain”)

Preferably, one of R₁ and R₂ is H.

Preferably, one of R₁ and R₂ is alkylcarbonyl (more preferably C₁-C₆ alkylcarbonyl), alkenylcarbonyl (more preferably C₂-C₆ alkenylcarbonyl), alkynylcarbonyl (more preferably C₂-C₆ alkynylcarbonyl). Even more preferably, one of R₁ and R₂ is C₁-C₆ alkylcarbonyl and most preferably methylcarbonyl (acetyl).

Preferably, R₃ is alkyloxy (more preferably C₁-C₆ alkyloxy), alkenyloxy (more preferably C₂-C₆ alkenyloxy), alkynyloxy (more preferably C₂-C₆ alkynyloxy) or aryloxy (more preferably phenyloxycarbonyl). Even more preferably, R₃ is benzyloxy.

Particularly preferred transforming agents are where R is H, R₂is acetyl and R₃ is carboxyl (N-acetyl glycine) or benzyloxy (N-acetyl glycine benzyl ester) and where R₁ and R₂ are H and R₃is benzyloxy (glycine benzyl ester). Particularly preferred transforming agents include glycine benzyl ester, glycylglycine ethyl ester, hippuric acid, p-amino hippuric acid and propargylglycine.

The method according to the invention is particularly suitable for increasing the sensitivity of a bacterial strain to an antimicrobial agent such as penicillin and its derivatives and analogues, in particular those that are stable to staphylococcal and similar β-lactamases (e.g. oxacillin), and to glycopeptides (e.g. vancomycin).

For the avoidance of doubt, the transforming agents useful in the method of the present invention include physiologically acceptable salts and other derivatives of the above-mentioned compounds of Formula I which are converted to a compound of formula I under physiological conditions.

It will be understood that said transforming agents generally do not in themselves have antimicrobial properties at ‘transforming’ levels, that is at concentrations which merely potentiate the activity of antimicrobial agents. Some of the compounds described may be antibacterial at higher levels, e.g. propargylglycine and hippuric acid.

Characteristics Associated with the Described Formula and its Variants

1. The term ‘transforming’ is exemplified by the transformation of a methicillin-resistant S. aureus to a methicillin-sensitive S. aureus.

2. Methicillin-resistance is not conferred by beta-lactamases. Where the staphylococcus is a beta-lactamase producer, the transforming agent will not influence sensitivity to antibiotics susceptible to beta-lactamases.

3. The action of the transforming agents extends to all staphylococci resistant to β-lactamase-resistant β-lactam antibiotics, including cephalosporins.

4. There is also activity against vancomycin-resistant enterococci (VRE), although the action is less potent. BTA activity in VRE is thought to be due to one or more glycine molecules within the cell wall cross-link(s) of these microorganisms.

5. The action of the transforming agents should extend to VRSA.

The present invention also resides in the use of an agent having formula (I) in the manufacture of a medicament for increasing the sensitivity of a bacterial strain infecting, colonising or being carried by a patient, to an antimicrobial agent. Preferably, said bacterial strain (i.e. the target of transformation) has resistance to s the antimicrobial agent to be co-formulated with the BTA.

The invention further resides in a method of prevention and/or treatment of infection of a patient by a carried bacterial strain, comprising administering to said patient an amount of a transforming agent of formula (I) sufficient to render said strain more sensitive to an antimicrobial agent, together with a therapeutically effective amount of said antimicrobial agent.

It will be understood that said patient may be a non-symptomatic carrier of the bacterial strain or said patient may be inflicted with a symptomatic clinical infection.

Administration of said transforming agent (BTA) may be prior to, subsequent to or concomitant with the administration of the antimicrobial agent. However, said transforming agent is preferably administered together with or prior to said antimicrobial agent. In the case of concomitant administration, the transforming agent and anti-microbial agent may be administered in combination as a single medicament or as separate medicaments. Preferably, the transforming agent and the antimicrobial agent are administered in combination as a single medicament (i.e. co-administered). It should be noted that the co-administered antimicrobial agent should have sufficient inherent activity against the species to which the target organism belongs, i.e. should have good activity against naturally sensitive variants of the resistant target organism.

Administration maybe by any known route eg. by intravenous, intramuscular, or intrathecal (spinal) injection, intranasal, topical administration as an ointment, salve, cream or tincture, oral administration as a tablet, capsule, suspension or liquid and nasal administration as a spray (eg. aerosol). The choice of administration route will be selected depending on the properties of the selected BTA.

In each case said agent or combination of agents may be in admixture with one or more excipients, carriers, emulsifiers, solvents, buffers, pH regulators, flavourings, colourings, preservatives, or other commonly used additives in the field of pharmaceuticals as appropriate for the mode of administration.

Preferably, said agent is capable of increasing the sensitivity to an appropriate cell-wall active antimicrobial agent of at least one bacterial strain selected from Staphylococcus aureus, coagulase-negative staphylococci, enterococci, Clostridium difficile and Streptococcus pneumoniae. More preferably, said agent is capable of increasing the sensitivity to the antimicrobial agent of at least one of methicillin-resistant Staphylococcus aureus and vancomycin-resistant enterococci, particularly where the bacterial stain is resistant to that antimicrobial agent, e.g. methicillin, oxacillin, flucloxacillin, vancomycin.. In particular, said agent is preferably capable of increasing the sensitivity of EMSRA-15,-16 and/or -17, or other EMRSA, to β-lactam (and analogous) antibiotics/antimicrobial agents, and/or increasing the sensitivity of EMSRA with reduced sensitivity to vancomycin, teicoplanin or other glycopeptide, or of VRSA to the aforementioned antimicrobial agents.

In each case, sensitivity is preferably increased to the level of a comparable non-resistant bacterial strain at a concentration of agent of 0.02M or less, more preferably 0.002M or less and most preferably 0.001M or less as determined by a standard antibiotic sensitivity test preferably the E-test.

Said agent is also capable of increasing the sensitivity of an already sensitive bacterial strain selected from Staphylococcus aureus, coagulase-negative staphylococci, enterococci, Clostridium difficile, Streptococcus pneumoniae, Streptococcus pyogenes and other streptococci and Gram-positive pathogens, to ‘hypersensitivity’ to a penicillin or analogue or derivative, or a glycopeptide. Said agent is therefore co-prescribable or maybe co-administered or co-formulated with an appropriate antimicrobial agent where the bacterial strain is causing a rapidly life-threatening infection, particularly in a debilitated host, to create ‘hypersensitivity’ of the infecting organisms to the antimicrobial agent.

Preferably, the anti-microbial agent to which sensitivity is increased is selected from the group consisting of β-lactam (and analogous) antibiotics (eg. methicillin, piperacillin, flucloxacillin, cloxacillin, oxacillin, Augmentin, ofloxacillin, imipenam and merpenam), cephalosporins (eg. ceftazidime and cefuroxime) and glycopeptides (eg. vancomycin, teicoplanin, gentamicin and oritavancin).

It will be understood that two or more antimicrobial agents (from the same or preferably different classes) may be employed.

Methicillin-Resistance in Staphylococci

The staphylococcal cell wall plays an important role in the pathogenesis and treatment of infection. In Grain-positive bacteria, the cell wall consists of layers of peptidoglycan that are cross-linked by peptide bridges. Gram-negative bacteria have a thin peptidoglycan layer encapsulated by an outer cell membrane. This peptidoglycan also contains cross-links and muropeptide tails that can be targeted by BTAs, as identified by the general principles outlined below. Because of the uniqueness of the peptidoglycan structure and assembly, it is one of the preferred targets of antimicrobial agents, including antibiotics produced naturally by several types of microorganisms. The peptidoglycan of Staphylococcus aureus consists of linear sugar chains of alternating units of N-acetylglucosamine and N-acetylmuramic acid substituted with a pentapeptide L-Ala-D-Glu-L-Lys-D-Ala-D-Ala. A characteristic of the cell wall of S. aureus is a pentaglycine cross-link that connects L-Lys to the D-Ala on the pentapeptide of a neighbouring unit, the terminal D-Ala being split off by transpeptidation. This flexible pentaglycine bridge allows up to 90% of the peptidoglycan units to be cross-linked, thus facilitating substantial cell-wall stability. In addition, the pentaglycine link acts as a recipient for staphylococcal surface proteins that are covalently anchored to it by a transpeptidase-like reaction. Surface proteins play an important role in adhesion and pathogenicity by interacting with host matrix proteins.

The major theory involving the mechanism of action of β-lactams concerns their structural similarity to the D-Ala-D-Ala carboxy-terminal region of the peptidoglycan pentapeptide. Penicillins, cephalosporins and other β-lactams, acylate the active site serine of cell wall transpeptidases, forming stable acylenzymes that lack catalytic activity. Inhibition of peptidoglycan synthesis by covalent binding of β-lactams to cell wall synthetic enzymes known as penicillin binding proteins (PBPs), allows autolysis in S. aureus mediated by endogenous autolytic enzymes. Although autolysis is less possible in MRSA, the llm gene encodes alipophillic protein of 351 amino acid residues that is associated with decreased methicillin resistance accompanied by increased autolysis. Methicillin-sensitive S. aureus produce four major PBPs with molecular masses of about 85, 81, 75 and 45 kDa, respectively referred to as PBPs 1, 2, 3 and 4 (by convention, PBPs are numbered in order of diminishing molecular mass). Resistance to penicillin in S. aureus was originally acquired in the form of β-lactamases or penicillinases, now produced by about 90% of clinical isolates. The structural gene for β-lactamase, blaZ, and two regulatory genes, blaI and blaRI, usually reside on a transmissible plasmid, although chromosomal location has been identified in some strains. The induction of β-lactamase is believed to be initiated by the binding of β-lactams to the transmembrane domain of a signal-transducing PBP encoded by blaRI (PBP3), leading ultimately to repressor degradation with loss of its DNA-binding properties, such that the transcription of blaZ is permitted. The means by which the BlaRI-penicillin complex causes repressor degradation is unclear, although it is thought that this could either result from, 1) conformational changes to BlaRI brought about by activation of a protease in the cytoplasmic domain by β-lactam binding, or 2) a repressor-inactivating protease encoded by a putative gene blaR2 which the BlaRI-penicillin complex either activates or causes to be induced. β-lactamases catalyse the inactivation of penicillin and other β-lactams (depending on the class of β-lactamase) by covalently binding to the β-lactam ring. This is essentially the same reaction that occurs when β-lactams bind to the active site of PBPs except that the reaction is non-hydrolytic and not reversible. Some PBPs have detectable β-lactamase activity, including PBP 4 of S. aureus. However, high molecular weight PBPs (eg. PBPs 1, 2 and 3 in S. aureus) are mainly involved with peptidoglycan transpeptidation, whilst low molecular weight ones exhibit carboxy peptidase activity.

Methicillin-resistance in S. aureus and coagulase-negative staphylococci is defined by the production ofa specific PBP, PBP2a, that has a reduced affinity for β-lactam compounds. The low affinity PBP2a, confers intrinsic resistance to virtually all β-lactam antimicrobial agents, including cephalosporins. PBP2a functions as a transpeptidase in cell wall synthesis in MRSA when high concentrations of β-lactams are present, which inhibits the activity of the normal PBPs, 14. PBP2a is encoded by the structural gene mecA located on the methicillin-resistant staphylococcal chromosome. Expression of PBP2a is controlled by two regulator genes on mec DNA, mecI and mecRI, located upstream of mecA, which encode a mecA repressor protein and signal transducer protein, respectively. MRSA carrying intact mecI and mecRI together with mecA, are referred to as ‘pre-MRSA’. Since intact mecI product strongly represses the expression of PBP2a, the pre-MRSA is apparently susceptible to methicillin. It has been hypothesised that removal of the repressor function for mecA is a prerequisite for constitutive expression of methicillin-resistance in S. aureus with mec DNA. There is homology between mecI and blaI, mecRI and blaRI, and the promoter and N-terminal portions of blaZ and mecA. This homology is strong enough that blaI can restore the normal inducible phenotype to isolates of S. aureus, which results in large amounts of constitutive PBP2a production because of the absence of or a defect in, the mecI locus. Increased PBP2a production may be associated with vancomycin-resistance (see below).

Subsequent to the discovery of PBP2a, it was realised that the phenotypic expression of methicillin-resistance did not correlate with the amount of PBP2a expressed. In 1983, it was shown that several additional genes independent of mecA are needed to sustain the high level of methicillin-resistance in MRSA. These genes were called fem, as they were thought to provide factors essential for methicillin-resistance, or aux for auxiliary factors. While it was originally thought that the fem or aux factors represented additional genes recruited by staphylococci after the acquisition of mecA to further improve and consolidate methicillin-resistance and its homogeneity, it became increasingly clear that the fem genes were natural constituents of all staphylococci, and were involved in the formation of the pentapeptide bridge and modification of this bridge or the muropeptide. Synthesis of the pentaglycine bridge occurs at the membrane-bound lipid II precursor NAG-(β-1,4)-NAM-(L-Ala-D-Glu-LLys-D-Ala-D-Ala)-pyrophosphoryl-undecaprenol by sequential addition of glycine to the ⊂-amino group of lysine, using glycyl-tRNA as donor, in a ribosome-independent fashion. Six fem genes (femA, femB, femC, femD, femE, femF) have been described. femA and femB are two closely related but distinct genes that form part of an operon. Both femA and femB have been shown to be involved with the formation of the pentaglycine bridge. FemA, the product of femA is responsible for adding glycines 2 and 3 to the bridge, whilst FemB, the product of femB, adds glycines 4 and 5. A hypothetical femX was proposed as being responsible for a protein that added the first glycine.

Other FemA,B-like factors were identified in staphylococci, such as Lif in Staphylococcus simulans and Eprin Staphylococcus capitis, which protect these organisms from their own glycyl-glycine endopeptidase. Three new genes fmhA, B and C, were subsequently identified. These fem-like genes are responsible for introducing 1-2 serine residues into the pentapeptide bridge in coagulase-negative staphylococci and may, under certain conditions, incorporate serine residues into positions 3 or 5 in the bridge in some strains of S. aureus. fmhB was subsequently shown to be the postulated femX, which added glycine residues to position 1 in the pentaglycine interpeptide bridge.

Inhibition of the formation of the pentaglycine bridge reduces resistance to methicillin without affecting synthesis of PBP2, resulting in β-lactam hyper-susceptibility (hyper-sensitivity). Thus the pentaglycine bridge has an important function in maintaining cell wall stability, including resistance to antimicrobial agents. This application also highlights the suitability of endogenous endopeptidases as the transforming target, because the natural activity of these enzymes can be harnessed to transform the sensitivity of bacterial cells to certain cell-wall active agents, as exemplified by the transformation of methicillin-resistant strains to methicillin-sensitive ones.

Vancomycin Resistance

Glycopeptide antibiotics are inhibitors of peptidoglycan synthesis. Unlike β-lactams and related antimicrobials, glycopeptides do not bind directly to cell wall biosynthetic enzymes (PBPs) but complex with the carboxy moiety of the terminal D-alanine of the cell wall precursor pentapeptide. This blocks progression to the subsequent transglycosylation steps in peptidoglycan synthesis and interferes with the reactions catalysed by D,D-transpeptidases and D,D-carboxypeptidases necessary for the anchoring of the peptidoglycan complex.

With the first appearance of VRE, it was apparent that strains could be divided by their type and level of glycopeptide resistance. There are now seven genotypic classes to characterise glycopeptide-resistant enterococci: vanA, found predominantly in E. faecium andE. faecalis that confers resistance to ≧256 mg/l of vancomycin and ≧32 mg/l of teicoplanin; vanB, found in E. faecium, E, faecalis and Streptococcus bovis that confers resistance to between 4 and 1000 mg/l of vancomycin and ≦1.0 of teicoplanin; vanC1 (E. gallinarium), vanC2 (E. casseliflavus), vanC3 (E. flavescens) that confers resistance to between 2 and 32 mg/l of vancomycin and ≦1.0 of teicoplanin; vanD, which confers resistance to between 64 and 256 mg/l of vancomycin and 4 to 32 mg/l of teicoplanin in E. faecium; and vanE, which confers resistance to 16 mg/l of vancomycin and 0.5 mg/l of teicoplanin in E. faecalis. VRE of VanA type provide the main model for achieving high-level vancomycin-resistance: instead of producing cell wall unit pentapeptides with D-Ala-D-Ala tails to which vancomycin and other glycopeptide's bind, the vanA gene cluster is induced by glycopeptides to produce D-Ala-D-Lac tails to which vancomycin and teicoplanin do not bind. The vain gene cluster is contained on a transposable element IN1546 and the vain gene itself produces a 39 Kda protein located in the cytoplasmio membrane. This protein is a ligase that preferentially synthesises D-Ala-D-Lac. In addition to vain, there are two other genes—vanH, which is a dehydrogenase enzymes that produces D-lac from pyruvate, and vanX, which encodes a metallo-dipeptidase that preferentially hydrolyses D-Ala-D-Ala. The trnscriptional activation of VanHAX is regulated by the VanRS two-component regulatory system comprising of the genes vanS, the signal sensor, and vanR, the response regulator. The remainder of the vanA gene cluster includes two additional genes, vanY (a D,D-carboxypeptidase that cleaves terminal D-Ala from pentapeptide residues and can increase the level of glycopeptide resistance further by eliminating binding targets, ie. D-Ala-DS-Ala) and vanZ (which mediates increased resistance to teicoplanin).

Tie ultimate emergence of vancomycin-resistant MRSA has been anticipated since it was shown experimentally that vanA genes from VRE maybe transferred into a recombinant-deficient S. aureus. However, this has not happened in practice with either S. aureus or coagulase-negative staphylococci. It appears that, in MRSA, vancomycin-tolerance does not occur without tolerance to β-lactams and that tolerant strains of S. aureus causing endocarditis, are associated with increased mortality. Vancomycin-tolerance has also emerged in Streptococcus pneumoniae and tolerant strains are more easily transformed to high-level resistance. This appears to be mediated by DNA changes in a two-component sensor-regulator system (VncS-VncR) which mediates changes in gene expression related to cell-wall formation. Amino-acid sequences of VncS and VncR show 38% homology to the VanS_{B}-VanR_{B} regulatory system associated with glycopeptide-resistance in vancomycin-resistant E. faecalis (VREF) and are probably relevant to MRSA. Indeed, over production of a 37 kd cytoplasmic protein thought to be a D-lactate dehydrogenase analogous to VanH in VREF, has been associated with vancomycin-resistance in a strain of S. aureus. This staphylococcal D-lactate dehydrogenase may also be under signal-transduction control mechanisms similar to the two-component homologous regions in S. pneumoniae and MRSA probably have sequences homologous to VanS_{B}-VanR_{B}/VncR-VncS. Vancomycin-resistance in MRSA has been achieved by other means rather than by the acquisition of new genetic elements, namely by altering cell wall composition, which is largely regulated by enzymes classically sensitive to penicillin (PBPs). Overproduction of PBP2a, a thickened cell wall containing a high glutamine non-amidated component, and an increase in cell wall synthesis have all been cited as mechanisms. The appearance ofa cell membrane dehydrogenase homologous to VanH in enterococci, has not yet been shown to be of importance in clinical strains, although there is a definite potential for high level vancomycin resistance to develop using this protein. Currently, the type of vancomycin-resistance encountered in S. aureus, has been described as Intermediate or reduced (sensitivity) which is usually difficult to detect by routine diagnostic methods. The main method of detection is by treatment failure. However, strains of VRSA have now been isolated in the USA and these are expected to spread world wide or mark the appearance of similar strains elsewhere.

Therapeutic use of teicoplanin is slightly controversial as it has not been approved for use in the USA and may select for vancomycin-resistant S. aureus. MRSA with reduced sensitivity to glycopeptides isolated from diabetic foot ulcers has been associated with use of teicoplanin and treatment failure has been associated with increased MICs of teicoplanin.

High concentrations of exogenous glycine are known to affect cell wall synthesis. Of more specific interest is the finding that glycine reduces the MIC of methicillin against MRSA: De Jonge and colleagues (Antimicrobial Agents and Chemotherapy(1996),40, pp1498-1503) used increasing concentrations of glycine in the growth medium, which resulted in peptidoglycan in which muropeptides with a D-Ala-D-Ala-terminus were replaced with D-Ala-glycine-terminating muropeptides. The authors concluded that the disappearance of D-Ala-D-Ala-terminating muropeptides in peptidoglycan and the concomitant decrease in resistance, indicated a central role for D-Ala-D-Ala-terminating precursors in methicillin resistance. It is believed that a significant effect of BTAs according to formula I is that the terminating muropeptide tail in staphylococci becomes D-Ala-BTA, and that this has transforming activity either alone or in conjunction with other effects, against methicillin and vancomycin resistance.

Initial experiments with MRSA prevalent in the UK during the 1980s found that 2% glycine transformed all MRSA into methicillin-sensitive strains. This occurred only in the presence of glycine; cells were not permanently affected. A more active agent, glycinebenzyl ester (GBE) was subsequently identified to produce transforming activity at levels of 0.1 to 1% In the presence of GBE, MRSA were also sensitive to cephalosporins and other β-lactam agents that were not hydrolysed by staphylococcal β-lactamase, i.e. penicillin-resistance was stable due to the production of this enzyme. The sensitivity achieved was commensurate with that achieved by these agents when tested against methicillin-sensitive strains, as has been discussed above. As far as the inventors are aware, the use of GBE as a transforming agent for the clinical treatment of MRSA has not been advanced. Nor has the use of GBE been investigated for the transformation of strains resistant to ‘non-β-lactam cell-wall active’ antimicrobials, for example glycopeptide antimicrobials.

The Following General Principles should be Followed for Identifying Transforming Agents in Microorganisms with Cell Walls

GBE is the first BTA with useful activity against which the potency of other compounds can be judged.

The method of identifying moieties is to establish the composition of cross-links in the cell wall of the target (i.e. chosen) organism, and test the transforming ability of the individual molecules against cell-wall active antimicrobials. Moieties that are repeated in any given cross-link are likely to indicate molecules with more useful potency. The chosen organisms will include infective microorganisms with cell-wall cross-links and dipeptide muropeptide tails, e.g. Gram-negative and Gram-positive bacteria, Chlamydia, etc.

Amino acid residues in cell wall cross-links are targeted by identical or structurally similar moieties contained within molecules that have greater potency than that achievable by the amino acids alone. Moieties of one or more amino acids in cell wall cross-links in structures that show increased potency over the transforming activity of the amino acid(s) alone.

In the case of MRSA, the cross-link is composed of five glycine molecules, for which N-acetyl glycine and glycine benzyl ester are the two stem BTA compounds. These basic BTAs demonstrate how molecules with a glycine moiety may expose the carboxylic or amino residues associated with the pentaglycine cross-link in S. In addition, endopeptidases such as the glycyl glycine endopeptidase ofstaphylococci may also be

In addition, endopeptidases as the glycyl glycine endopeptidase of staphylococci may also be potential transforming targets, because the natural activity ofthese enzymes canbe harnessed to transform the sensitivity ofbacterial cells to certain cell-wall active agents, as exemplifiedby the transformation ofmethicillin-resistant strains to methicillin-sensitive ones. The precise molecular interactions ofthe BTAs described in this application is not known, but interaction with glycyl glycine dipeptidases and other enzymes involved with the formation and remodelling ofcell wall cross-links and muropeptide tails, are most probable.

It is also the purpose ofthis application to prescribe asimilarapproach to identifyingBTAs specific to vancomycin resistance, which in VRE and VRSA is based on the alteration of cell wall muropeptide tails from D-Ala-D-Ala to D-Ala-D-Lac or other variations. BTAs could therefore have moieties of D-Ala-D-Lac or other variations or be able to directly replace the terminal amino acid to fonn D-Ala-BTA tails. The screening ofsuch compounds for transforming activity should follow the methods described in this application.

Thus, it is also the purpose ofthis application to direct the development of all molecules that interact with cross-links and muropeptide tails in the cell walls of microorganisms of medical importance, either directly or indirectly in amanner similar to that established by GBE, such that these organisms are transformed to a clinically relevant susceptibility, i.e. one that is treatable by a suitable cell-wall-active antimicrobial agent co-prescribed or co-adniinistered with the BTA.

### EXAMPLES

To find substances related to GBE that might have increased potency, various substances, including those with additional glycine moieties and benzylates, have been screened. Screening was carried out using Isosensitest agar (Oxoid, UK) into which various levels of potential BTAs were incorporated at levels between 0.01 and 1.0%. The agar with incorporated BTA was then used in the manner of a standard antibiotic sensitivity test using 10 μg methicillin discs. The test organism was inoculated onto the agar surface at a concentration suitable to achieve confluent growth after 18 hours incubation at 30° C. After incubation, zone diameters were compared with that achieved by the control plate (Isosensitest alone) for each test organism.

Glycine Benzyl Ester (GBE) [C₉H₁₁NO₂] (Comparative Example)

Glycine t-butyl ester [C₇H₇NO₄] (Example 1)

Glycine anhydride [C₄H₆N₂O₂] (Example 2)

Glycine ethyl ester [C₄H₉NO₂] (Example 3)

N,N-Dimethylglycine [(CH₃)₂NCH₂CO₂H] (Example 4)

N,N-Dimethylglycine ethyl ester [(CH₃)₂NCH₂CO₂C₂H₅] (Example 5)

Glycine methyl ester [C₃H₇NO₂] (Example 6)

Di-glycine (glycylglycine) [C₄H₈N₂O₃] (Example 7)

Glycylglycine methyl ester [C₅H₁₀N₂O₃] (Example 8)

Glycylglycine ethyl ester [C₆H₁₂N₂O₃] (Example 9)

Glycylglycine benzyl ester [C₁₁H₁₄N₂O₃] (Example 10)

Triglycine [C₆H₁₁N₃O₄] (Example 11)

N-acetylglycine (NAGly) [C₆H₁₂N₂O₃] (Example 12)

N-tris(hydroxymethyl)methyl glycine [C₆H₁₃NO₅] (Example 13)

N,N-di-methyl glycine [C₄H₉NO₂] (Example 14)

D-2-(t-butyl) glycine [C₆H₁₃NO₅] (Example 15)

Glycinamide [C₂H₆N₂O] (Example 16)

N-carbamoylglycine (Hydantoic acid) [C₃H₆N₂O₃] (Example 17)

N-CBZ-glycine (C₁₀H₁₁NO₄] (Example 18)

N-Phthaloylglycine (1,3-dioxo-2-isoindolineacetic acid) [C₁₀H₇NO₄] (Example 19)

N-(2-Mercaptopropionyl) glycine [CH₃CH(SH)CONHCH₂] (Example 20)

N-(2-Carboxyphenyl) glycine [HO₂CC₆H₄NHCH₂CO₂H] (Example 21)

N-(2-Furoyl) glycine [C₇H₇NO₄] (Example 22)

N-(2-Furoyl) glycine methyl ester [C₈H₉NO₄] (Example 23)

1-Amino-1-cyclopropanecarboxylic acid [C₄H₇NO₂] (Example 24)

Propargylglycine (2-Amino-4-pentynoic acid) [C₅H₇NO₂] (Example 25)

2-Phenylglycine [C₆H₅CH(NH₂)CO₂H] (Example 26)

2-Phenylglycine methyl ester [C₆H₅CH(NH₂)CO₂CH₃] (Example 27)

N-(2-Carboxyphenyl)glycine [HO₂CC₆H₄NHCH₂CO₂H] (Example 28)

D-4-Hydroxyphenylglycine [HOC₆H₄CH(NH₂)CO₂H] (Example 29)

N-(4-Hydroxyphenyl)glycine [HOC₆H₄NHCH₂CO₂H] (Example 30)

2,2-Diphenylglycine [H₂NC(C₆H₅)₂CO₂H] (Example 31)

Hippuric acid (N-Benzoylglycine) [C₉H₈NO₃] (Example 32)

2-Methylhippuric acid [CH₃C₆H₄CONHCH₂CO₂H] (Example 33)

3-Methylhippuric acid [CH₃C₆H₄CONHCH₂CO₂H] (Example 34)

4-Methylhippuric acid [CH₃C₆H₄CONHCH₂CO₂H] (Example 35)

P-Amino Hippuric acid [C₉H₉N₂O₃] (Example 36)

2-Iodohippuric acid [C₆H₄CONHCH₂CO₂H] (Example 37)

Arg-Gly [C₈H₁₇N₅O₃] (Example 38)

All the above substances, including glycine itself transformed a reference MRSA (type strain) and various selected MRSA (OMRSA), EMRSA-1 and EMRSA-16.

Hydantoic acid had low-level active against vancomycin-resistant enterococci (VRE) whereas GBE and glycylglycine ethyl ester have greater activity against VRE and MRSA than glycylglycine benzyl ester. P-Amino Hippuric acid has improved activity compared to Hippuric acid and GBE.

Different salts may have altered activity and stability, as may other analogues, including peptide, benzylate, amino and acelate variants and extended compounds.

Table 1 shows the improved effect on methicillin sensitivity of glycinebenzyl ester (GBE) (Example 10) on various patient isolated MRSA (L-series) and reference strains. At the time of isolation, the patient isolates were resistant to all clinically available β-lactams, cephalosporins, macrolides and gentamicin There was variable sensitivity to tetracycline, trimethoprim, chloramphenicol, fusidic acid and rifampicin.

As can be seen from Table 1, glycine benzyl ester increased sensitivity to methicillin to a much greater extent than glycine. Even at 0.001M, an improved effect was observed over glycine at 0.2M (test 3 cf. test 1) for all strains.

TABLE 1

| | MIC of methicillin (mg/l) | | | |
|---|---|---|---|---|
| | | Glycine | | |
| | | 0.02M | | GBE |
| Isolate | 0.0 | (0.15%) | 0.2M (1.5%) | 0.001M (0.2%) |
| tested | (Control) | (Test 1) | (Test 2) | (Test 3) |
| NCTC 12493 | >256 | 0.12 | 0.06 | 0.015 |
| L265 | >256 | 64 | 8 | 2 |
| L266 | >256 | 64 | 8 | 2 |
| L267 | >256 | 32 | 8 | 2 |
| L268 | >256 | 32 | 8 | 2 |
| L269 | >256 | 16 | 4 | 1 |
| L270 | >256 | 8 | 4 | 1 |
| L271 | >256 | 8 | 4 | 2 |
| L272 | >256 | 16 | 2 | 1 |
| L273 | >256 | 8 | 4 | 1 |
| L274 | >256 | 16 | 4 | 2 |
| L275 | >256 | 32 | 8 | 2 |
| L276 | >256 | 16 | 4 | 2 |
| L277 | >256 | 8 | 4 | 2 |
| L278 | >256 | 64 | 16 | 4 |
| L279 | >256 | 64 | 2 | 2 |
| L280 | >256 | 16 | 4 | 2 |
| L281 | >256 | 32 | 4 | 2 |
| L282 | >256 | 32 | 4 | 2 |
| L283 | >256 | 32 | 4 | 2 |
| L284 | >256 | 32 | 2 | 2 |
| L285 | >256 | 32 | 4 | 2 |
| L286 | >256 | 32 | 4 | 2 |
| L287 | >256 | 32 | 4 | 2 |
| L288 | >256 | 32 | 4 | 2 |
| L289 | >256 | 32 | 4 | 2 |
| L290 | >256 | 32 | 4 | 2 |
| L291 | >256 | 32 | 4 | 2 |
| L292 | >256 | 32 | 4 | 2 |
| L293 | >256 | 32 | 4 | 2 |
| L294 | >256 | 32 | 4 | 2 |
| MC01* | >256 | 32 | 4 | 2 |
| JF1-32* | >256 | 32 | 4 | 2 |
| DS09* | >256 | 32 | 4 | 2 |
| SW2-32* | >256 | 32 | 4 | 2 |
| PS3-32* | >256 | 32 | 4 | 2 |
| ST11* | >256 | 32 | 4 | 2 |
| SN31* | >256 | 32 | 4 | 2 |
| CD40* | >256 | 32 | 4 | 2 |
| E16-96** | >256 | 32 | 4 | 2 |
| E15-97*** | >256 | 32 | 4 | 2 |

*EMRSA-1;
**EMRSA-16;
***EMRSA-15

In table 1, the target MIC for transformation is provided by the vancomycin-sensitive reference strain NCTC 12493, which has an MIC of vancomycin of 2 mg/l. 0.2 M glycine achieves this target in 50% ofstrains tested, compared to 0.02 M of glycylbenzyl ester which achieves complete transformation in 100% of strains tested.

Importantly, the usefulness of the agents of the present invention is not limited to increasing bacterial sensitivity to methicillin. The transforming effect of glycyl benzyl ester on two cephalosporins is shown in Table 2.

TABLE 2

| Isolate | MIC of ceftazidime or cefuroxime (mg/l) when grown | | | |
|---|---|---|---|---|
| of | with or without glycine benzyl ester (GBE): | | | |
| MRSA | Control | | GBE (0.2%) | |
| tested | Ceftazidime | Cefuroxime | Ceftazidime | Cefuroxime |
| NCTC 12493 | >256 | >256 | 2 | 4 |
| MC01* | >256 | >256 | 2 | 4 |
| JF1-32* | >256 | >256 | 2 | 2 |
| DS09* | >256 | >256 | 2 | 2 |
| SW2-32* | >256 | >256 | 4 | 4 |
| PS3-32* | >256 | >256 | 4 | 4 |
| ST11* | >256 | >256 | 2 | 2 |
| SN31* | >256 | >256 | 4 | 4 |
| CD40* | >256 | >256 | 4 | 2 |
| E16-96** | >256 | >256 | 2 | 4 |
| E15-97*** | >256 | >256 | 4 | 4 |

*EMRSA-1;
**EMRSA-16;
***EMRSA-15

Glycyl benzyl ester transforms the MRSA tested to ceftazidime and cefuroxime sensitivity, thus making these two drugs that have never had useful activity against MRSA newly active against MRSA.

The potential for useful activity in vivo, is demonstrated in Table 3, which shows the MICs of methicillin in 1% human plasma for 19 patient-isolates ofMRSA for glycine benzyl ester and glycine as a reference. Stored frozen plasma was pooled from five subjects.

TABLE 3

| | MIC of methicillin (mg/l) when grown | |
|---|---|---|
| | in Moles (%) of glycine or GBE | |
| | with or without 1% human plasma | |
| Isolate | Glycine | GBE |
| of | (0.02M [0.15%]) | (0.00075M [0.15%]) |

| MRSA | No plasma | +plasma | No plasma | +plasma |
|---|---|---|---|---|
| tested | (Control 1) | (Test 1) | (Control 2) | (Test 2) |
| L277 | 8 | 32 | 4 | 8 |
| L278 | 64 | 256 | 8 | 16 |
| L279 | 64 | 256 | 4 | 16 |
| L280 | 16 | 64 | 4 | 8 |
| L281 | 8 | 32 | 4 | 8 |
| L282 | 32 | 256 | 4 | 16 |
| L283 | 32 | 128 | 4 | 16 |
| L284 | 32 | 256 | 2 | 8 |
| L285 | 32 | 256 | 4 | 16 |
| L286 | 32 | 64 | 4 | 8 |
| L287 | 32 | 128 | 4 | 16 |
| L288 | 32 | 128 | 2 | 16 |
| L289 | 32 | 256 | 4 | 16 |
| L290 | 32 | 128 | 4 | 8 |
| L291 | 32 | 64 | 4 | 16 |
| L292 | 32 | 256 | 4 | 32 |
| L293 | 32 | 128 | 2 | 8 |
| L294 | 16 | 128 | 2 | 8 |
| 5518* | 8 | 32 | 2 | 4 |

*EMRSA-1

Human plasma may bind or otherwise inactivate foreign substances and good activity in plasma is indicative of good in vivo activity. Approximations from the above data suggest glycine is reduced in activity by about 75% and glycine benzyl ester by about 75% to 50%. This may be due to protein binding rather than enzymatic degradation, indicating the useful stability of the compound in vivo. Again the increase in sensitivity to methicillin is significantly increased for glycine benzyl ester relative to glycine.

Table 4 shows the ability of glycine benzyl ester and N-acetyl glycine (NAGly) (Example 4) to transform MRSA with intermediate resistance to glycopeptides into glycopeptide-sensitive strains.

TABLE 4

| | MIC of vancomycin | | |
|---|---|---|---|
| | or teicoplanin (mg/l) | | |
| | when grown in Moles (%) | | |
| | of GBE or NAGly of: | | |
| MRSA | | NAGly | GBE |
| tested | Control | 0.001M | 0.001M |
| MICs of vancomycin | | | |
| EMRSA-17 (VISA) | | | |
| L266 | 8 | 4 | 2 |
| L266 | 8 | 2 | 1 |
| NCTC 12493 | 0.5 | 0.25 | 0.12 |
| MICs of teicoplanin | | | |
| EMRSA-16 (TISA) | | | |
| L265 | 32 | 4 | 1 |
| L266 | 8 | 4 | 2 |
| NCTC 12493 | 0.25 | 0.15 | 0.06 |

This data shows that glycine benzyl ester and N-acetyl glycine can restore the activity of vancomycin in vancomycin-intermediate-resistant MRSA (VISA) and teicoplanin in teicoplanin-intermediate-resistant MRSA (TISA), by reducing MICs to below the recognised resistant threshold of an MIC of 8 mg/I which defines intermediate resistance, at very low concentrations (0.001M).

The agents of the present invention are not limited to the reversal of resistance in Staphylococcus. The test strains in Table 5 are patient-isolates of vancomycin- and gentamicin-resistant Enterococcus faecium. At the time of isolation, they were commonly resistant to all clinically useable antimicrobial agents.

TABLE 5

MIC of vancomycin (mg/l) when
grown in Moles (%) of glycine
or glycine benzyl ester (GBE) of:

| | |
|---|---|
| Glycine | GBE |

| Strain | 0.0 | 0.02M | 0.2M | 0.02M |
|---|---|---|---|---|
| tested | (Control) | (Test 1) | (Test 2) | (Test 3) |
| ATCC 29212 | 2 | 4 | 2 | 1 |
| S317 | 128 | 32 | 4 | 2 |
| S227 | 128 | 32 | 4 | 2 |
| E267 | 128 | 16 | 4 | 2 |
| E254 | 128 | 16 | 4 | 2 |
| E297 | 128 | 8 | 2 | 2 |
| S226 | 128 | 8 | 4 | 2 |
| S283 | 64 | 8 | 2 | 2 |
| S315 | 64 | 4 | 1 | 1 |
| S497 | 64 | 8 | 2 | 1 |
| E285 | 64 | 16 | 2 | 2 |
| S556 | 64 | 32 | 2 | 1 |
| S319 | 64 | 16 | 4 | 2 |
| S302 | 64 | 8 | 4 | 2 |
| S393 | 64 | 8 | 2 | 2 |
| E271 | 64 | 8 | 2 | 2 |
| S333 | 64 | 8 | 2 | 2 |
| GBC | 64 | 8 | 4 | 2 |
| WBC | 64 | 8 | 4 | 2 |
| BBC | 32 | 16 | 4 | 2 |
| S337 | 32 | 4 | 2 | 2 |

In table 5, the target MIC for transformation is provided by the vancomycin-sensitive reference strain ATCC 29212, which has an MIC of vancomycin of 2 mg/l. 0.2M glycine achieves this target in 50% of strains tested, compared to 0.02 M of GBE which achieves complete transformation in 100% of strains tested.

As previously mentioned, a common cause of auto-infection is due to S. aureus carried on the anterior nares. The data in Table 6 show that glycyl benzyl ester increases the sensitivity of already sensitive bacteria to methicillin (and by implication other related antibiotics such as flucloxacillin). The transforming agents of the present invention may also be used in combination with a suitable antimicrobial to eliminate nasal carriage of MSSA prior to cardiac surgery or other invasive procedures carrying a high risk of auto-infection.

TABLE 6

MIC of methicillin (mg/l) when grown in
Moles (%) of glycine or GBE with or
without 1% human plasma

GBE (0.00075M [0.15%])

| | | No plasma | |
|---|---|---|---|
| Isolate | No plasma | (Test 1 and | plasma (1%) |
| tested | (Control 1) | control 2) | (Test 2) |
| LHS77 | <0.25 | <0.25 | <0.25 |
| LHS78 | 0.25 | <0.25 | 1 |
| LHS79 | 0.5 | <0.25 | 0.25 |
| LHS80 | 0.25 | <0.25 | 0.25 |
| LHS81 | <0.25 | <0.25 | <0.25 |
| LHS82 | 0.5 | <0.25 | 4 |
| LHS83 | 0.25 | <0.25 | 0.25 |
| LHS84 | 0.25 | <0.25 | 2 |
| LHS85 | 0.25 | <0.25 | 0.5 |
| LHS86 | 0.25 | <0.25 | 0.25 |
| LHS87 | 0.5 | <0.25 | 4 |
| LHS88 | 0.25 | <0.25 | 0.5 |
| LHS89 | 0.25 | <0.25 | 0.5 |
| LHS90 | <0.25 | <0.25 | <0.25 |
| LHS91 | <0.25 | <0.25 | <0.25 |
| LHS92 | 0.5 | <0.25 | 1 |
| LHS93 | 0.25 | <0.25 | 0.5 |
| LHS94 | 0.25 | <0.25 | 0.5 |
| 5518* | >256 | <0.25 | 0.5 |

*EMRSA-1

Table 7 demonstrates the activity of five BTA compounds according to the present invention. The four clinical isolates were isolated from patients during the first three months of 2003. The latest isolates have been used because they represent strain evolution, particularly in epidemic MRSA, exemplified by their greater ability to produce reduced sensitivity to glycopeptides. An intermediate MRSA has been included, as methicillin-resistance has been achieved by means other than production of PBP2a. The reduced sensitivity of EMRSA-17 to vancomycin is transformed by the BTAs, as is resistance to cephalexin, which is normally minimally active against staphylococci.

TABLE 7

| Antimicrobial | | | | |
|---|---|---|---|---|
| plus BTA | I-MRSA | EMRSA-16 | EMRSA-17 | VRE |
| Oxacillin | 32 | 256 | 256 | N/A |
| +GBE 1.0% | 0.75 | 1.0 | 1.5 | N/A |
| +GBE 0.1% | 1.0 | 2 | 2 | N/A |
| +GBE 0.01% | 2 | 4 | 6 | N/A |
| +GGEE 1.0% | 0.32 | 0.75 | 1.0 | N/A |
| +GGEE 0.1% | 0.75 | 1.0 | 2 | N/A |
| +GGEE 0.01% | 1.0 | 1.5 | 3 | N/A |
| +HA 0.1% | 2 | 4 | 4 | N/A |
| +HA 0.01% | 4 | 6 | 8 | N/A |
| +Amino-HA 0.1% | 0.064 | 0.75 | 1.0 | N/A |
| +Amino-HA 0.01% | 1.5 | 3 | 6 | N/A |
| +PPG 0.01% | 0.125 | 1.5 | 2 | N/A |
| +PPG 0.001% | 2.0 | 4 | 8 | N/A |
| Vancomycin | 0.5 | 1.0 | 2 | >256 |
| +GBE 1.0% | <0.25 | 0.25 | 0.25 | 64 |
| +GGEE 1.0% | <0.25 | 0.25 | 0.25 | 64 |
| +HA 0.1% | <0.25 | 0.25 | 0.25 | 64 |
| +Amino-HA 0.1% | <0.25 | 0.25 | 0.25 | 64 |
| +PPG 0.01% | <0.25 | 0.25 | 0.25 | 64 |
| Cephalexin* | 32 | 256 | 256 | N/A |
| +GBE 1.0% | 0.75 | 1.0 | 1.5 | N/A |
| +GBE 0.1% | 1.0 | 2 | 2 | N/A |
| +GGEE 1.0% | 0.38 | 0.75 | 1.0 | N/A |
| +GGEE 0.1% | 0.75 | 1.5 | 2 | N/A |
| +HA 0.1% | 2 | 6 | 8 | N/A |
| +Amino-HA 0.1% | 0.064 | 1.0 | 4 | N/A |
| +Amino-HA 0.01% | 1.5 | 3 | 6 | N/A |
| +PPG 0.01% | 0.125 | 12 | 6 | N/A |

I-MRSA = Intermediate MRSA;
EMRSA = epidemic MRSA;
GBE = glycine benzyl ester;
GGEE = glycyl glycine ethyl ester;
PPG = propargylglycine (2-amino-4-pentynoic acid);
HA = hippuric acid;
Amino-HA = P-amino hippuric acid
*= not active against VRE

For clinical use, the agents may be administered systemically (eg. intravenously) for serious systemic infections such as septicaemia. However, it is anticipated that one of the principle uses of the agents will be topical administration for the subsequent treatment of local infections, or as part of a program to eliminate resistant bacteria from a carrier prior to surgery, for example, to prevent dissemination of infection before it arises.

The following is a non-exhaustive list of antibiotics which may be incorporated with the transforming agents of the present invention and their preferred routes of administration:-

Oral administration: flucloxacillin, cloxacillin, oxacillin, piperacillin

IV administration: vancomycin, meropenem, flucloxacillin, cloxacillin, oxacillin, piperacillin, cefuroxime.

IM administration: flucloxacillin, cefuroxime, ceftriaxone.

Topical: flucloxacillin, oxacillin, cefalexin

General Formulation Considerations

As far as systematic administration is concerned, co-formulation is generally preferred if the half-lives of the transforming agent and the antimicrobial are comparable. For example the penicillins generally have a half life of about 1.5 to 2 hrs and are administered 3 to 4 times daily. On the other hand teicoplanin has a half life of 12 hrs and is usually administered once a day. Thus, the transforming agent should be selected to have a corresponding half life, or alternatively be administered separately on a different dosing regimen.

In general, the transforming agent should be in sufficient concentration to achieve in vivo levels that will effect transformation in the target bacteria during approximately the same period as the halflife of the antimicrobial. Of course it will be understood that the actual concentration of the transforming agent is not relevant to the concentration of the antimicrobial in the formulation. It will also be understood that where the target organism is a bacterial strain which has evolved from an original progenitor, it is essential that the co-formulated or co-administered antibiotic has demonstrably useful activity against the original progenitor strain of the target organism(s). This is a necessary requirement as the transforming agent completely or partly reduces the resistance of the evolved target organism, maximally to that of a sensitive equivalent strain.

### Medicament Example 1

Glycine benzyl ester, glycylglycine ethyl ester, hippuric acid, P-amino hippuric acid or propargyiglycine) and flucloxacillin or oxacillin, are mixed with paraffin wax, softisan [TM], hydroxypropyl methyl cellulose, polyglyceryl-4-caprate and glycerine to give an ointment containing 0.2 wt % of the BTA and 1 wt % of flucloxacillin or oxacillin.

Treatment Regime

The ointment is rubbed into the infected area 3 to 4 times daily until the infection is eliminated, or applied to a deep wound at dressing. This medication may also be applied to the insertion site of intravascular devices as a prophylactic measure against cannula- or catheter-related infection.

### Medicament Example 2

N-acetyl glycine or one of the BTAs listed in Table 7 and cefuroxime or oxacillin or other suitable antimicrobial agent, are mixed with an inert carrier liquid to give a 1% w/v of each active and dosed to a spray applicator.

Treatment Regime

The medicament is sprayed intranasally 3 to 4 times daily for five days prior to surgery (or during a hospital outbreak) to eliminate anterior nares carriage of S. aureus. Treatment can be continued after surgery if desired or if there is re-inoculation of the carriage site.

The spray may also be used to administer the antimicrobial product to a surgical wound before closure to prevent infection (e.g. sternal wounds; bone and joint prosthesis or grafts).

The spray may also be used to administer the antimicrobial product to chronic ulcers (e.g. diabetic foot ulcers) before dressing or if the ulcer is being left open.

### Medicament Example 3

A 1.0% solution of a BTA (e.g. as in Table 7) plus a suitable antimicrobial agent such as oxacillin or cefuroxime, are made up in a solution, e.g. normal saline.

Treatment Regime for a Vascular Graft

The vascular graft is placed in the solution and left to soak, prior to implantation.

1. A method of increasing the sensitivity of a bacterial strain to a cell-wall active antimicrobial agents, to which the bacterial strain or a progenitor strain from which the bacterial strain has evolved is sensitive, said method comprising the step of exposing said bacterial strain to at least one transforming agent having the following formula (I):- 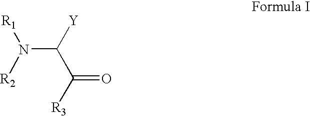
where
moieties R₁ and R₂ are each independently selected from, alkyl, alkyloxy, alkyloxycarbonyl, alkylcarbonyloxy, alkenyl, alkenyloxy, alkenyloxycarbonyl, alkenylcarbonyloxy, alkynyl, alkynyloxy, alkynyloxycarbonyl, alkynylcarbonyloxy, each of which may be substituted or unsubstituted, straight chain or branched or cyclic, aryl, aryloxy, aryloxycarbonyl, arylcarbonyloxy, each of which may be substituted or unsubstituted, and
carbamoyl,
moiety R₃ is selected from alkyl, alkyloxy, alkylcarbonyloxy, alkenyl, alkenyloxy, alkenylcarbonyloxy, alkynyl, alkynyloxy, alkynylcarbonyloxy, each of which may be substituted or unsubstituted, straight chain or branched or cyclic, aryl, aryloxy, arylcarbonyloxy, each of which may be substituted or unsubstituted, and carboxyl.
other than R₁, R₂, and R₃ are not all H,
and Y is selected from a natural amino acid side chain,
or a physiologically acceptable salt or derivative thereof which is converted to a compound of formula I under physiological conditions.

2. A method as claimed in claim 1 wherein Y is —H₂ (i.e. glycine “side chain”).

3. A method as claimed in claim 1 wherein one of R₁ and R₂ is H.

4. A method as claimed in claim 1 wherein one of R₁ and R₂ is alkylcarbonyl (more preferably C₁-C₆ alkylcarbonyl), alkenylcarbonyl (more preferably C₂-C₆ alkenylcarbonyl), alkynylcarbonyl (more preferably C₂-C₆ alkynylcarbonyl).

5. A method as claimed in claim 1 wherein one of R₁ and R₂ is C₁-C₆ alkylcarbonyl and preferably methylcarbonyl (acetyl).

6. A method as claimed in claim 1 wherein R₃ is alkyloxy (more preferably C₁-C₆ alkyloxy), alkenyloxy (more preferably C₂-C₆ alkenyloxy), alkynyloxy (more preferably C₂-C₆ alkynyloxy) or aryloxy (more preferably phenyloxycarbonyl).

7. A method as claimed in claim 1 wherein R₃ is benzyloxy.

8. A method as claimed in claim 1 wherein the antimicrobial agent is penicillin, a cephalosporin or a derivative or analogue thereof or a glycopeptide.

9. A method as claimed in claim 8 wherein the antimicrobial agent is a β-lactamase-stable penicillin or a derivative or analogue thereof.

10. A method as claimed in claim 1 wherein the antimicrobial agent is oxacillin or vancomycin.

11. A method as claimed in claim 1 wherein the transforming agent is glycine benzyl ester, glycylglycine ethyl ester, hippuric acid, p-amino hippuric acid or propargylglycine.

12. The use of an agent having formula (I) of claim 1 in the manufacture of a medicament for increasing the sensitivity of a bacterial strain infecting, colonising or being carried by a patient, to a cell-wall active antimicrobial agent as described in claim 1.

13. A method of preventing infection and cross-infection related to carriage of a bacterial strain, comprising topical administration to the carriage site(s) of said patient, an amount of a transforming agent of formula (I) of claim 1, sufficient to render said strain more sensitive to an antimicrobial agent and administering to said patient a therapeutically effective amount of said antimicrobial agent as a co-formulant and/or co-administrant.

14. A method as claimed in claim 13 wherein said agent may be in admixture with one or more excipients, carriers, emulsifiers, solvents, buffers, pH regulators, flavourings, colourings, preservatives, or other commonly used additives in the field of pharmaceuticals as appropriate for the mode of administration.

15. A method as claimed in claim 13 wherein said agent is capable of increasing the sensitivity to the antimicrobial agent of at least one bacterial strain selected from Staphylococcus aureus, coagulase-negative staphylococci and enterococci, Clostridium difficile, and Streptococcus pneumoniae and other Gram-positive pathogens.

16. A method as claimed in claim 15 wherein the bacterial strain is resistant to the antimicrobial agent.

17. A method as claimed in claim 13 wherein said agent is capable of increasing the sensitivity to the antimicrobial agent of at least one of methicillin- and/or glycopeptide-resistant Staphylococcus aureus and vancomycin-resistant enterococci to the antimicrobial agent to which the bacterial strain is resistant.

18. A method as claimed in claim 17 wherein the bacterial strain is resistant to at least one of methicillin and its derivatives or related antimicrobial agents; vancomycin, teicoplanin or another related glycopeptides.

19. A method as claimed in claim 13 wherein said agent is capable of increasing the sensitivity to the antimicrobial agent of at least one bacterial strain selected from Staphylococcus aureus, coagulase-negative staphylococci, enterococci, Clostridium difficile, Streptococcus pneumoniae, Streptococcus pyogenes and other streptococci and Gram-positive pathogens, where the bacterial strain is causing a rapidly life-threatening infection, particularly in a debilitated host, to create ‘hypersensitivity’ of the infecting organisms to the antimicrobial agent.

20. A method as claimed in claim 13 wherein said agent is capable of increasing the sensitivity of EMSRA-15, -16 and/or -17, or other EMRSA, to β-lactam (and analogous) antibiotics/antimicrobial agents, and/or increasing the sensitivity of EMSRA with reduced sensitivty to vancomycin, teicoplanin or other glycopeptide, or of VRSA to the aforementioned antimicrobial agents.

21. A method as claimed in claim 19 wherein sensitivity is increased to the level of a comparable non-resistant bacterial strain at a concentration of agent of 0.02M or less, more preferably 0.002M or less and most preferably 0.001M or less as determined by a standard antibiotic sensitivity test.

22. A method as claimed in claim 13 wherein the antimicrobial agent to which sensitivity is increased is selected from the group consisting of β-lactam (and analogous) antibiotics/antimicrobial agent stable to staphylococcal β-lactamases), cephalosporins and glycopeptides.

23. A method as claimed in claim 22 wherein the antimicrobial agent to which sensitivity is increased is methicillin, flucloxacillin, cloxacillin, oxacillin, imipenam, meropenam, ceftazidime, cefuroxime, vancomycin, teicoplanin or oritavancin.

24. A method as claimed in claim 13 wherein the antimicrobial agent to which sensitivity is increased is selected from those agents that consist of a β-lactam (and analogous) antibiotics/antimicrobial agent sensitive to β-lactamases, together with a β-lactamase inhibitor or a derivative or analogue thereof.

25. A method for identifying transforming agents in microorganisms of medical importance with cell walls of the structure suitable for targeting by penicillin and related/analogous antimicrobial agents and glycopeptides, wherein the composition of cross-links and muropeptide tails in the cell wall of the target organism must be wholly or partly established, and the transforming ability of the individual molecules with corresponding moieties selected for testing.
